(19) United States
(12) Patent Application Publication
(10) Pub. No.: US 2020/0165266 A1
(43) Pub. Date: May 28, 2020

(54) RING CLOSURE REACTION

(71) Applicant: MERCK PATENT GMBH, Darmstadt (DE)

(72) Inventors: Changsheng WANG, Durhum (GB); William MITCHELL, Chandler's Ford (GB)

(73) Assignee: FLEXENABLE LIMITED, Cambridge (GB)

(21) Appl. No.: 16/630,198

(22) PCT Filed: Jul. 9, 2018
(86) PCT No.: PCT/EP2018/068463

§ 371 Date: Jan. 10, 2020

(30) Foreign Application Priority Data
Jul. 12, 2017 (EP) 17180869.4

(51) Int. Cl. C07D 495/22; C07D 495/04

(52) U.S. Cl. CPC C07D 495/22; C07D 495/04

(57) ABSTRACT

The present invention relates to a ring closure reaction useful in synthesizing fused aromatic or heteroaromatic ring systems, which may, for example, be used as organic semiconductor materials.

### TECHNICAL FIELD

The present invention relates to a ring closure reaction useful in synthesizing fused aromatic or heteroaromatic ring systems, which may, for example, be used as organic semiconductor materials.

### BACKGROUND

Organic electronic and optoelectronic applications, such as for example, organic field effect transistors (OTFTs), organic light-emitting diodes (OLEDs), organic photodetectors (OPDs) or organic photovoltaic cells (OPVs), require semiconducting organic compounds comprising aromatic or heteroaromatic ring systems, preferably fused (i.e. polycyclic) aromatic or heteroaromatic ring systems. An exemplary class of such structures comprises conjugated arylene/heteroarylene rings that are interlocked or bridged via sp³-carbon atoms. A specific example of such a structure is 1,5-dihydro-s-indaceno[3,2b; 7,6b]dithiophene (IDT), which is a remarkable building block leading to outstanding semiconducting organic materials of high electronic performance (X. Zhang, et al., Nat. Commun., 2013, 4, 2238).

However, the synthesis of such fused aromatic or heteroaromatic ring systems may be rather challenging. It generally requires multi-step reactions, some steps of which may have to be conducted under harsh reaction conditions and/or make use of toxic reactants, and on top of all, frequently result in low overall yields (W. Zhang, et al., J. Am. Chem. Soc., 2010, 132, 11437-11439). While in the research stage it is generally possible to synthesize such ring systems in sufficient quantity, for example, on the scale of 1 g or 10 g or even 100 g, it is often found that the upscaling of such reactions is difficult particularly because of the high toxicity of some of the reactants, purification issues and/or low reaction yields in one or more of the synthetic steps.

One of the critical steps in the synthesis of fused aromatic or heteroaromatic ring systems is the ring closure reaction that forms one or more of the sp³-carbon atom bridges. It is desirable that such ring closure succeeds, preferably under mild and benign reaction conditions, in high yield and/or purity and/or also employs readily available raw materials, preferably without using toxic and/or dangerous reactants.

Aromatically fused 1,5-dihydro-s-indacenes have previously been synthesized by different multi-step reaction routes. In one of these, the dicarboxylic acid precursors are converted to the corresponding diketones via an intramolecular Friedel-Crafts reaction followed by Wolf-Kishner reduction using hydrazine, which is highly toxic (W. Zhang, et al., J. Am. Chem. Soc., 2010, 132, 11437-11439). The other multi-step reaction route starts from phenylene bis(tert-alcohol) derivatives to conduct acid catalyzed ring-closure reactions. Ring closure reactions of biphenyl alcohol derivatives of the following formula (where R═H or Acetyl)

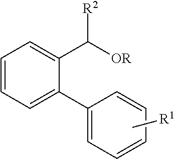

using Bronsted/Lewis acids are, for example, disclosed by G. Li et al. in Tetrahedron, 2008, 64, 9033-9043. However, these reactions demand either R¹ to be a strongly electron donating methoxy group and R² to be an aromatic group. Li et al. did not observe any reaction for both, R¹ and R², being H, thereby limiting the scope of compounds, particularly, aromatically fused multi-ring structures, such as 1,5-dihydro-s-indacene, that can be prepared by this method.

It is therefore an object of the present application to provide a ring closure reaction that does not have the above disadvantages/limitations and can be readily used on a commercial scale to make fused aromatic or heteroaromatic ring systems. Preferably such ring closure reaction will give the desired product in good yield or good purity or both.

In a particular aspect, the present application is directed to a simplified method for preparing fused cyclopentadiene ladder structures where the sp³ carbon atoms are unsubstituted —CH₂— groups. These structures are key precursors for achieving symmetric alkylations.

### SUMMARY OF THE INVENTION

The present inventors have now surprisingly found that the above objects may be attained either individually or in any combination by the process of the present application.

The present application therefore provides for a process of reacting a reactant comprising two adjacent moieties Ar¹ and Ar², wherein
(i) Ar¹ and Ar² are linked by a carbon-carbon single bond;
(ii) Ar¹ and Ar² are at each occurrence independently selected from the group consisting of arenes, arenes substituted with R^{S}, heteroarenes and heteroarenes substituted with R^{S}, with R^{S} being a halogen or a carbyl group; and
(iii) one of Ar¹ and Ar² has a group of formula —CH₂—OH in ortho-position to said carbon-carbon single bond,
   in the presence of a strong acid to obtain a product, wherein the Ar¹ and Ar² are fused to a five or six-membered ring, which comprises said carbon-carbon single bond and the formed CH₂-bridge

Additionally, the present application provides for the compound obtained by said process.

### DETAILED DESCRIPTION OF THE INVENTION

For the purposes of the present application, the term “arene” is used to denote a monocyclic or polycyclic aromatic hydrocarbon.

For the purposes of the present application, the term “heteroarene” is used to denote heterocyclic compounds formally derived from arenes by replacement of one or more methine (—CH═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hückel rule (4n+2). See also International Union of Pure and Applied Chemistry, Compendium of Chemical Technology, Gold Book, Version 2.3.3, 2014-02-24, page 671.

For the purposes of the present application, the term “aromatic” is used to denote a cyclically conjugated molecular entity with a stability (due to electron delocalization) significantly greater than that of a hypothetical localized structure.

For the purposes of the present application, the terms “triflic acid” and “trifluoromethanesulfonic acid” are used interchangeably.

In a very general sense the present process is directed to reacting a reactant comprising two moieties Ar¹ and Ar², one of which bears a hydroxymethyl group at the ortho position, in the presence of a strong acid in such a way that a new five- or six-membered ring is formed between Ar¹ and Ar², with Ar¹ and Ar² being fused to this newly formed five- or six-membered ring. In other words, the present process relates to a ring closure reaction, wherein a five- or six-membered ring is formed. It is noted that in the present process the strong acid may, but does not need to, serve as both, reactant and solvent.

Preferably the strong acid is selected from the group consisting of triflic acid, polyphosphoric acid, fluorosulfuric acid, SbF₅, BF₃, and any mixture comprising or consisting of one or more of these acids. Most preferably, the strong acid is triflic acid.

In the reactant, Ar¹ and Ar² are linked by a carbon-carbon single bond. For reasons of clarity it is noted that Ar¹ and Ar² are comprised in the same molecule. It is also noted that the carbon-carbon single bond directly connects Ar¹ and Ar².

In the reactant, one of Ar¹ and Ar² has a group —CH₂—OH in ortho-position to the carbon-carbon single bond linking Ar¹ and Ar².

Preferably, the reactant comprises one or more structural units of formula (I)

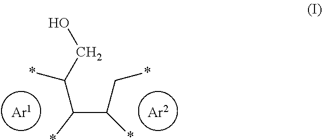

with Ar¹ and Ar² as defined herein. The corresponding product will then comprise one or more structural units of formula (II), which may generally be described as a diareno-cyclopentadiene.

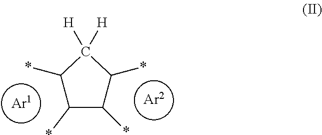

The present process is also very well suited to perform more than one ring closure reactions essentially simultaneously in the same reactant, i.e. performing more than one ring closure reactions essentially simultaneously within the same reactant molecule. Exemplary reactants capable of performing more than one ring closure reaction essentially simultaneously may be selected from the group consisting of the following formulae (I-A), (I-B) and (I-C), and preferably is of formula (I-B) (wherein both —CH₂—OH groups are on Ar²)

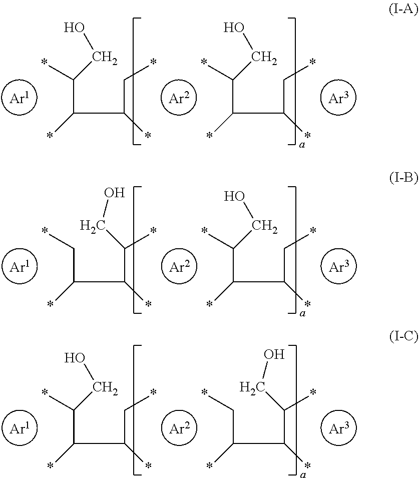

with Ar¹ and Ar² at each occurrence independently as defined herein, Ara at each occurrence independently defined as for Ar¹ and Ar², a being an integer selected from the group consisting of 1, 2, 3, 4 and 5, and wherein adjacent —CH₂—OH groups may be cis or trans to one another. It is also noted that for a >1, subsequent units bearing the —CH₂—OH groups may be oriented either way as schematically indicated in the following formulae

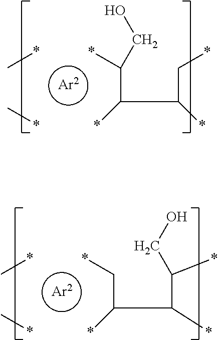

For the purposes of the present application the terms “cis” and “trans” are used to indicate the relative orientation of adjacent —CH₂—OH groups to each other. Examples of cis-configuration are schematically shown in formulae (I-A′) to (I-C′) below. Examples of trans-configuration are schematically shown in formulae (I-D′) to (I-F′) below.

Examples of reactants capable of two simultaneous ring closure reactions are schematically shown in formulae (I-A′) to (I-F′) and the corresponding products in formulae (II-A′) and (II-B′)

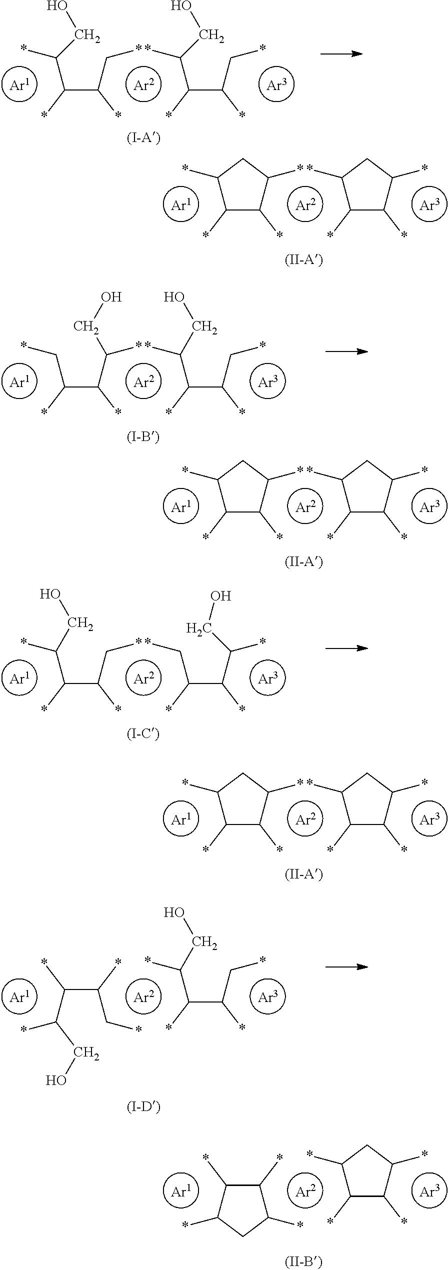

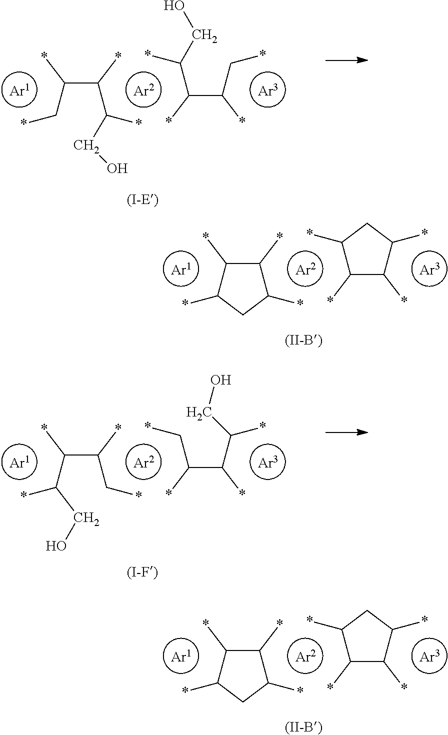

with Ar¹ and Ar² as defined herein and Ar³ defined as for Ar¹ and Ar². Due to the different orientation and location of the —CH₂—OH groups, the present process also allows the synthesis of a wide range of products, for example products wherein the newly formed five-membered rings are in a cis- or trans-orientation with respect to each other, as schematically shown in formulae (II-A′) and (II-13′), respectively. Products corresponding to formulae (II-A′) and (II-13′) may generally be described as diareno-dihydroindacene derivatives.

In the reactant, Ar¹ and Ar² and—if present—Ar³ are at each occurrence independently selected from the group consisting of arenes and heteroarenes. Preferably Ar¹ and Ar² and—if present—Ar³ are at each occurrence independently of each other selected from the group consisting of the following formulae (III-1) to (III-11)

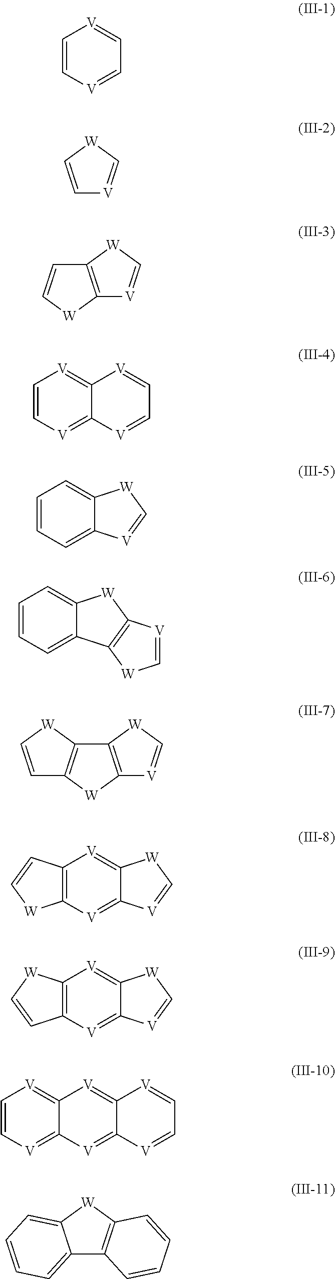

which may optionally be substituted by one or more group R^{S}, and wherein W is at each occurrence independently selected from the group consisting of S, O and Se; and V is at each occurrence independently CR^{S} or CR⁰ or N, with R^{S} in this case including H.

With regards to formulae (I) and (II), it is preferred that at least one of Ar¹ and Ar² is selected from the group consisting of (III-1), (III-4) and (III-10), and most preferably is of formula (III-1), and V is preferably CR^{S}, with R^{S} in this case being preferably selected from the group consisting of H, F, alkyl having from 1 to 10, preferably from 1 to 5, carbon atoms, such alkyl may also be fully or partially fluorinated, and alkoxy having from 1 to 10, preferably from 1 to 5, carbon atoms, R^{S} being more preferably selected from the group consisting of H, F and alkyl having from 1 to 10, preferably from 1 to 5, carbon atoms, and most preferably R^{S} being H or F.

With regards to formulae (I-A), (I-B) and (I-C), Ar¹ and Ar³ are independently of each other—though preferably they are identical—preferably selected from the group consisting of formulae (III-1), (III-2), (III-3), (III-4) and (III-10), more preferably selected from the group consisting of formulae (III-1), (III-2) and (III-3), wherein W—if present—is preferably S, and/or V—if present—is preferably CR^{S}, with R^{S} in this case being preferably selected from the group consisting of H, F, alkyl having from 1 to 10, preferably from 1 to 5, carbon atoms, such alkyl may also be fully or partially fluorinated, and alkoxy having from 1 to 10, preferably from 1 to 5, carbon atoms, R^{S} being more preferably selected from the group consisting of H, F and alkyl having from 1 to 10, preferably from 1 to 5, carbon atoms, and most preferably R^{S} being H or F.

With regards to formula (I-A), (I-B) and (I-C), Ar² is preferably selected from the group consisting of (III-1), (III-2), (III-3), (III-4) and (III-10), more preferably selected from the group consisting of formulae (III-1), (III-4) and (III-10), and most preferably is of formula (III-1), wherein W—if present—is preferably S, and/or V—if present—is—is preferably CR^{S}, with R^{S} in this case being preferably selected from the group consisting of H, F, alkyl having from 1 to 10, preferably from 1 to 5, carbon atoms, such alkyl may also be fully or partially fluorinated, and alkoxy having from 1 to 10, preferably from 1 to 5, carbon atoms, R^{S} being more preferably selected from the group consisting of H, F and alkyl having from 1 to 10, preferably from 1 to 5, carbon atoms, and most preferably R^{S} being H or F.

R^{S} is at each occurrence independently a halogen, with fluorine being the preferred halogen, or a carbyl group as defined herein and is preferably selected from the group consisting of any group R^{T} as defined herein, hydrocarbyl having from 1 to 40 carbon atoms wherein the hydrocarbyl may be further substituted with one or more groups R^{T}, and hydrocarbyl having from 1 to 40 carbon atoms comprising one or more heteroatoms selected from the group consisting of N, O, S, P, Si, Se, As, Te or Ge, with N, O and S being preferred heteroatoms, wherein the hydrocarbyl may be further substituted with one or more groups R^{T}.

Preferred examples of hydrocarbyl suitable as R^{S} may at each occurrence be independently selected from phenyl, phenyl substituted with one or more groups R^{T}, alkyl and alkyl substituted with one or more groups R^{T}, wherein the alkyl has at least 1, preferably at least 5 and has at most 40, more preferably at most 30 or 25 or 20, even more preferably at most 16 and most preferably at most 12 carbon atoms. It is noted that, for example, alkyl suitable as R^{S} also includes fluorinated alkyl, i.e. alkyl wherein one or more hydrogen is replaced by fluorine, and perfluorinated alkyl, i.e. alkyl wherein all of the hydrogen are replaced by fluorine.

In particular, R^{S} may be selected from the group consisting of fluorine, alkyl having at least 1, preferably at least 5 and has at most 40, more preferably at most 30 or 25 or 20, even more preferably at most 16 and most preferably at most 12 carbon atoms, and partially or fully fluorinated alkyl having at least 1, preferably at least 5 and having at most 40, more preferably at most 30 or 25 or 20, even more preferably at most 16 and most preferably at most 12 carbon atoms,

R^{T} is at each occurrence independently selected from the group consisting of F, Br, Cl, —CN, —NC, —NCO, —NCS, —OCN, —SCN, —C(O)NR⁰R⁰⁰, —C(O)X⁰, —C(O)R⁰, —NH₂, —NR⁰R⁰⁰, —SH, —SR⁰, —SO₃H, —SO₂R⁰, —OH, —OR⁰, —NO₂, —SF₅ and —SiR⁰R⁰⁰R⁰⁰⁰. Preferred R^{T} are selected from the group consisting of F, Br, Cl, —CN, —NC, —NCO, —NCS, —OCN, —SCN, —C(O)NR⁰R⁰⁰, —C(O)X⁰, —C(O)R⁰, —NH₂, —NR⁰R⁰⁰, —SH, —SR⁰, —OH, —OR⁰ and —SiR⁰R⁰⁰R⁰⁰⁰. Most preferred R^{T} is F.

R⁰, R⁰⁰ and R⁰⁰⁰ are at each occurrence independently of each other selected from the group consisting of H, F, hydrocarbyl having from 1 to 40 carbon atoms, and hydrocarbyl having from 1 to 40 carbon atoms wherein one or more hydrogen have been replaced by F. Said hydrocarbyl preferably has at least 5 carbon atoms. Said hydrocarbyl preferably has at most 30, more preferably at most 25 or 20, even more preferably at most 20, and most preferably at most 12 carbon atoms. Preferably, R⁰, R⁰⁰ and R⁰⁰⁰ are at each occurrence independently of each other selected from the group consisting of H, F, alkyl, fluorinated alkyl, alkenyl, alkynyl, phenyl and fluorinated phenyl. More preferably, R⁰, R⁰⁰ and R⁰⁰⁰ are at each occurrence independently of each other selected from the group consisting of H, F, alkyl, fluorinated, preferably perfluorinated, alkyl, phenyl and fluorinated, preferably perfluorinated, phenyl.

It is noted that, for example, alkyl suitable as R⁰, R⁰⁰ and R⁰⁰⁰ also includes perfluorinated alkyl, i.e. alkyl wherein all of the hydrogen are replaced by fluorine. Examples of alkyls suitable as R⁰, R⁰⁰ and R⁰⁰⁰ may be selected from the group consisting of methyl, ethyl, n-propyl, iso-propyl, n-butyl, iso-butyl, tert-butyl (or “t-butyl”), pentyl, hexyl, heptyl, octyl, nonyl, decyl, undecyl, dodecyl, tridecyl, tetradecyl, pentadecyl, hexadecyl, heptadecyl, octadecyl, nonadecyl and eicosyl (—C₂₀H₄₁).

X⁰ is halogen. Preferably X⁰ is selected from the group consisting of F, Cl and Br.

A hydrocarbyl group comprising a chain of 3 or more carbon atoms and heteroatoms combined may be straight chain, branched and/or cyclic, including spiro and/or fused rings.

Hydrocarbyl suitable as R^{S}, R⁰, R⁰⁰ and/or R⁰⁰⁰ may be saturated or unsaturated. Examples of saturated hydrocarbyl include alkyl. Examples of unsaturated hydrocarbyl may be selected from the group consisting of alkenyl (including acyclic and cyclic alkenyl), alkynyl, allyl, alkyldienyl, polyenyl, aryl and heteroaryl.

Preferred hydrocarbyl suitable as R^{S}, R⁰, R⁰⁰ and/or R⁰⁰⁰ include hydrocarbyl comprising one or more heteroatoms and may for example be selected from the group consisting of alkoxy, alkylcarbonyl, alkoxycarbonyl, alkylcarbonyloxy and alkoxycarbonyloxy, alkylaryloxy, arylcarbonyl, aryloxycarbonyl, arylcarbonyloxy and aryloxycarbonyloxy.

Preferred examples of aryl and heteroaryl comprise mono-, bi- or tricyclic aromatic or heteroaromatic groups that may also comprise condensed rings.

Especially preferred aryl and heteroaryl groups may be selected from the group consisting of phenyl, phenyl wherein one or more CH groups are replaced by N, naphthalene, fluorene, thiophene, pyrrole, preferably N-pyrrole, furan, pyridine, preferably 2- or 3-pyridine, pyrimidine, pyridazine, pyrazine, triazole, tetrazole, pyrazole, imidazole, isothiazole, thiazole, thiadiazole, isoxazole, oxazole, oxadiazole, thiophene, preferably 2-thiophene, selenophene, preferably 2-selenophene, thieno[3,2-b]thiophene, thieno[2,3-b]thiophene, dithienothiophene, furo[3,2-b]furan, furo[2,3-b]furan, seleno[3,2-b]selenophene, seleno[2,3-b]selenophene, thieno[3,2-b]selenophene, thieno[3,2-b]furan, indole, isoindole, benzo[b]furan, benzo[b]thiophene, benzo[1,2-b; 4,5-b′]dithiophene, benzo[2,1-b; 3,4-b′]dithiophene, quinole, 2-methylquinole, isoquinole, quinoxaline, quinazoline, benzotriazole, benzimidazole, benzothiazole, benzisothiazole, benzisoxazole, benzoxadiazole, benzoxazole and benzothiadiazole.

Preferred examples of an alkoxy group, i.e. a corresponding alkyl group wherein the terminal CH₂ group is replaced by —O—, can be straight-chain or branched, preferably straight-chain (or linear). Suitable examples of such alkoxy group may be selected from the group consisting of methoxy, ethoxy, propoxy, butoxy, pentoxy, hexoxy, heptoxy, octoxy, nonoxy, decoxy, undecoxy, dodecoxy, tridecoxy, tetradecoxy, pentadecoxy, hexadecoxy, heptadecoxy and octadecoxy.

Preferred examples of alkenyl, i.e. a corresponding alkyl wherein two adjacent CH₂ groups are replaced by —CH═CH— can be straight-chain or branched. It is preferably straight-chain. Said alkenyl preferably has 2 to 10 carbon atoms. Preferred examples of alkenyl may be selected from the group consisting of vinyl, prop-1-enyl, or prop-2-enyl, but-1-enyl, but-2-enyl or but-3-enyl, pent-1-enyl, pent-2-enyl, pent-3-enyl or pent-4-enyl, hex-1-enyl, hex-2-enyl, hex-3-enyl, hex-4-enyl or hex-5-enyl, hept-1-enyl, hept-2-enyl, hept-3-enyl, hept-4-enyl, hept-5-enyl or hept-6-enyl, oct-1-enyl, oct-2-enyl, oct-3-enyl, oct-4-enyl, oct-5-enyl, oct-6-enyl or oct-7-enyl, non-1-enyl, non-2-enyl, non-3-enyl, non-4-enyl, non-5-enyl, non-6-enyl, non-7-enyl, non-8-enyl, dec-1-enyl, dec-2-enyl, dec-3-enyl, dec-4-enyl, dec-5-enyl, dec-6-enyl, dec-7-enyl, dec-8-enyl and dec-9-enyl.

Especially preferred alkenyl groups are C₂-C₇-1E-alkenyl, C₄-C₇-3E-alkenyl, C₅-C₇-4-alkenyl, C₆-C₇-5-alkenyl and C₇-6-alkenyl, in particular C₂-C₇-1E-alkenyl, C₄-C₇-3E-alkenyl and C₅-C₇-4-alkenyl. Examples of particularly preferred alkenyl groups are vinyl, 1E-propenyl, 1E-butenyl, 1E-pentenyl, 1E-hexenyl, 1E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl, 6-heptenyl and the like. Alkenyl groups having up to 5 C atoms are generally preferred.

Preferred examples of oxaalkyl, i.e. a corresponding alkyl wherein one non-terminal CH₂ group is replaced by —O—, can be straight-chain or branched, preferably straight chain. Specific examples of oxaalkyl may be selected from the group consisting of 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl and 2-, 3-, 4-, 5-, 6-, 7-, 8- or 9-oxadecyl.

Preferred examples of carbonyloxy and oxycarbonyl, i.e. a corresponding alkyl wherein one CH₂ group is replaced by —O— and one of the thereto adjacent CH₂ groups is replaced by —C(O)— may be selected from the group consisting of acetyloxy, propionyloxy, butyryloxy, pentanoyloxy, hexanoyloxy, acetyloxymethyl, propionyloxymethyl, butyryloxymethyl, pentanoyloxymethyl, 2-acetyloxyethyl, 2-propionyloxyethyl, 2-butyryloxyethyl, 3-acetyloxypropyl, 3-propionyloxypropyl, 4-acetyloxybutyl, methoxycarbonyl, ethoxycarbonyl, propoxycarbonyl, butoxycarbonyl, pentoxycarbonyl, methoxycarbonylmethyl, ethoxy-carbonylmethyl, propoxycarbonylmethyl, butoxycarbonylmethyl, 2-(methoxycarbonyl)ethyl, 2-(ethoxycarbonyl)ethyl, 2-(propoxycarbonyl)ethyl, 3-(methoxycarbonyl)propyl, 3-(ethoxycarbonyl)propyl, and 4-(methoxycarbonyl)-butyl.

Preferred examples of thioalkyl, i.e where one CH₂ group is replaced by —S—, may be straight-chain or branched, preferably straight-chain. Suitable examples may be selected from the group consisting of thiomethyl (—SCH₃), 1-thioethyl (—SCH₂CH₃), 1-thiopropyl (—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) and 1-(thiododecyl).

A fluoroalkyl group is preferably perfluoroalkyl C₁F₂ᵢ₊₁, wherein i is an integer from 1 to 15, in particular CF₃, C₂F₅, C₃F₇, C₄F₉, C₅F₁₁, C₆F₁₃, C₇F₁₅ or C₈F₁₇, very preferably C₆F₁₃, or partially fluorinated alkyl, in particular 1,1-difluoroalkyl, all of which are straight-chain or branched.

Alkyl, alkoxy, alkenyl, oxaalkyl, thioalkyl, carbonyl and carbonyloxy groups can be achiral or chiral groups. Particularly preferred chiral groups are 2-butyl (=1-methylpropyl), 2-methylbutyl, 2-methylpentyl, 3-methylpentyl, 2-ethylhexyl, 2-propylpentyl, 2-butyloctyl, 2-hexyldecyl, 2-octyldodecyl, 7-decylnonadecyl, in particular 2-methylbutyl, 2-methylbutoxy, 2-methylpentoxy, 3-methylpentoxy, 2-ethyl-hexoxy, 1-methylhexoxy, 2-octyloxy, 2-oxa-3-methylbutyl, 3-oxa-4-methylpentyl, 4-methylhexyl, 2-butyloctyl, 2-hexyldecyl, 2-octyldodecyl, 7-decylnonadecyl, 3,8-dimethyloctyl, 2-hexyl, 2-octyl, 2-nonyl, 2-decyl, 2-dodecyl, 6-meth-oxyoctoxy, 6-methyloctoxy, 6-methyloctanoyloxy, 5-methylheptyloxy-carbonyl, 2-methylbutyryloxy, 3-methylvaleroyloxy, 4-methylhexanoyloxy, 2-chloropropionyloxy, 2-chloro-3-methylbutyryloxy, 2-chloro-4-methyl-valeryl-oxy, 2-chloro-3-methylvaleryloxy, 2-methyl-3-oxapentyl, 2-methyl-3-oxa-hexyl, 1-methoxypropyl-2-oxy, 1-ethoxypropyl-2-oxy, 1-propoxypropyl-2-oxy, 1-butoxypropyl-2-oxy, 2-fluorooctyloxy, 2-fluorodecyloxy, 1,1,1-trifluoro-2-octyloxy, 1,1,1-trifluoro-2-octyl, 2-fluoromethyloctyloxy for example. Most preferred is 2-ethyl hexyl.

Preferred achiral branched groups are isopropyl, isobutyl (=methylpropyl), isopentyl (=3-methylbutyl), tert. butyl, isopropoxy, 2-methyl-propoxy and 3-methylbutoxy.

In a preferred embodiment, the organyl groups are independently of each other selected from primary, secondary or tertiary alkyl or alkoxy with 1 to 30 C atoms, wherein one or more H atoms are optionally replaced by F, or aryl, aryloxy, heteroaryl or heteroaryloxy that is optionally alkylated or alkoxylated and has 4 to 30 ring atoms. Very preferred groups of this type are selected from the group consisting of the following formulae

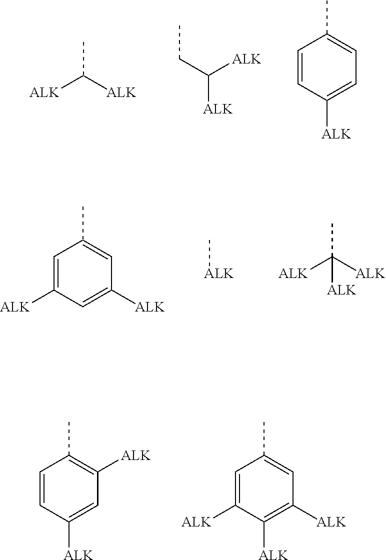

wherein “ALK” denotes optionally fluorinated, preferably linear, alkyl or alkoxy with 1 to 20, preferably 1 to 12 C-atoms, in case of tertiary groups very preferably 1 to 9 C atoms, and the dashed line denotes the link to the ring to which these groups are attached. Especially preferred among these groups are those wherein all ALK subgroups are identical.

The molar ratio of triflic acid to the number of —CH₂OH groups comprised in the reactant is preferably at least 1, more preferably at least 5, even more preferably at least 10 and most preferably at least 15.

The present process is performed at a temperature of preferably at most 50′C (for example at most 45° C. or 40° C. or 35° C. or 30° C. or 25° C. or 20° C. or 15° C. or 10° C.).

Preferred examples of reactants, products and the corresponding reaction may be selected from the group consisting of the following, which optionally may be substituted with R^{S}

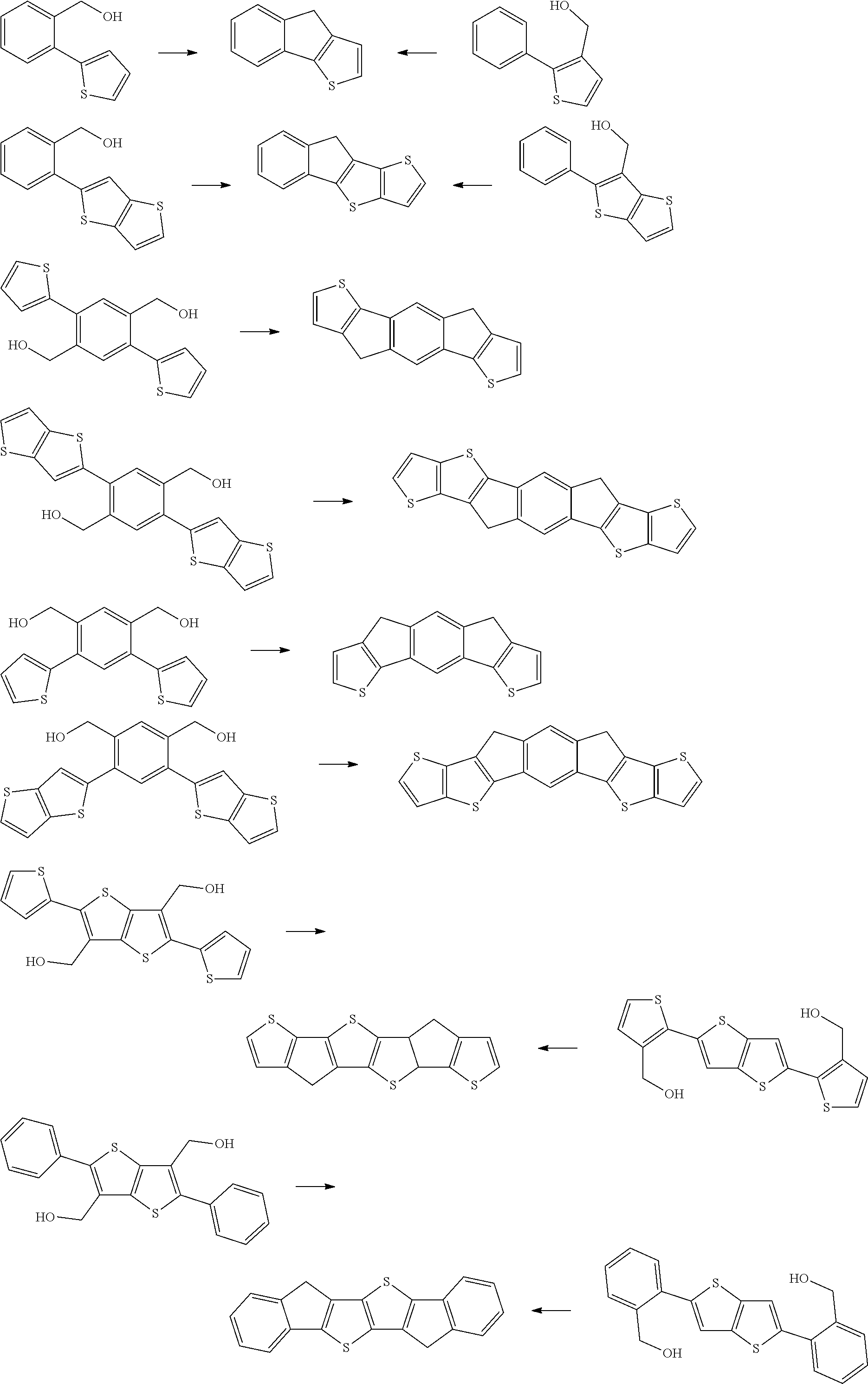

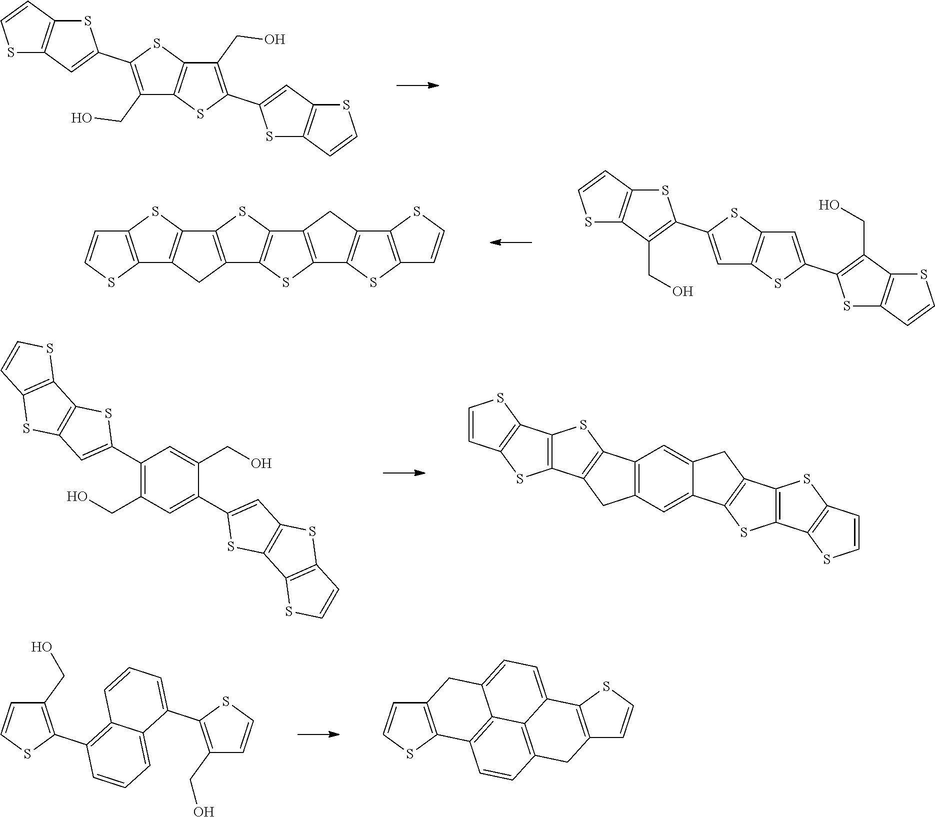

Very preferred examples of reactants, products and the corresponding reaction may be selected from the group consisting of the following, which optionally may be substituted with R^{S}

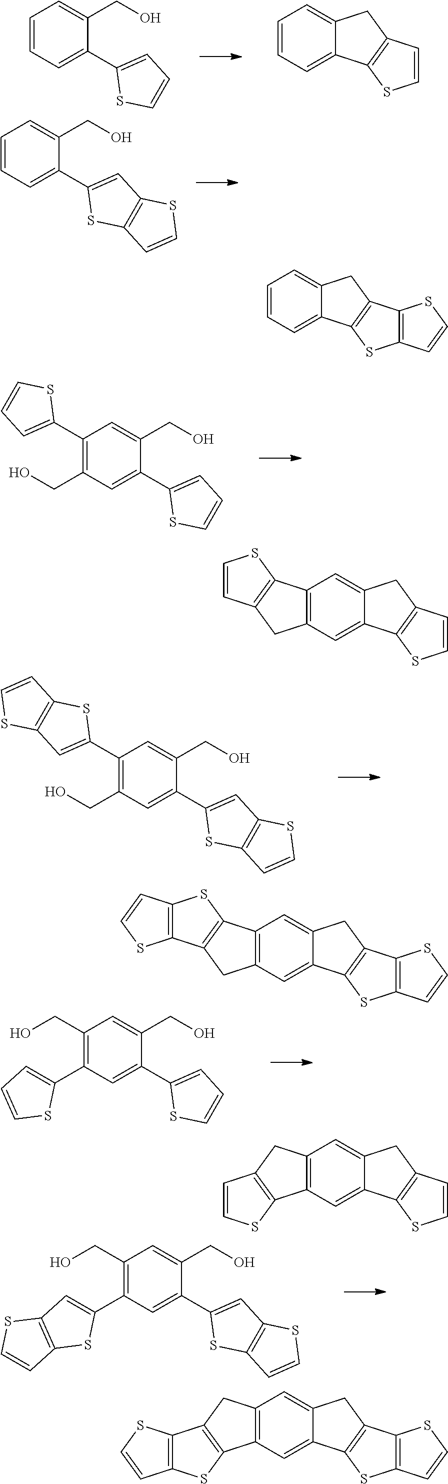

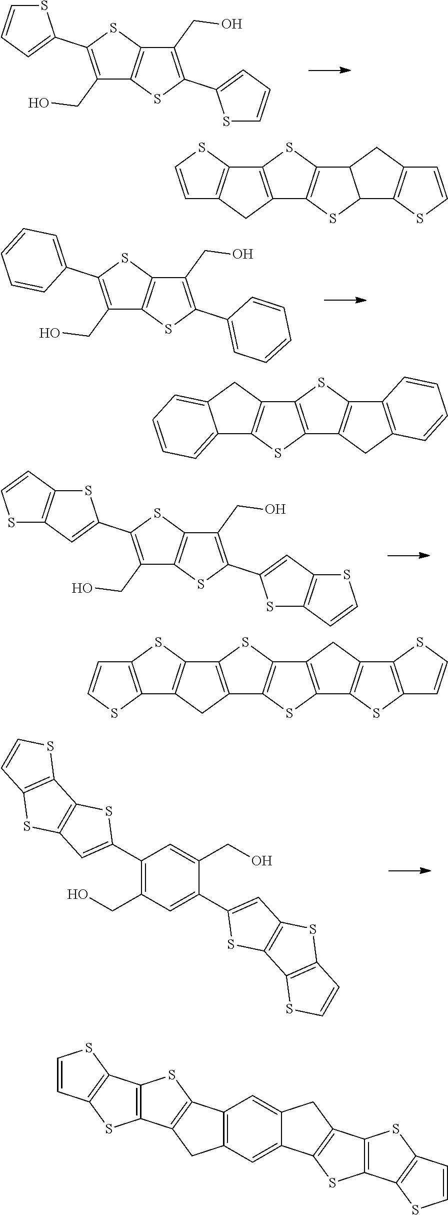

Most preferred examples of reactants (left column), products (right column) and the corresponding reaction may be selected from the group consisting of the following, wherein it is most preferred that R^{S} is H or F

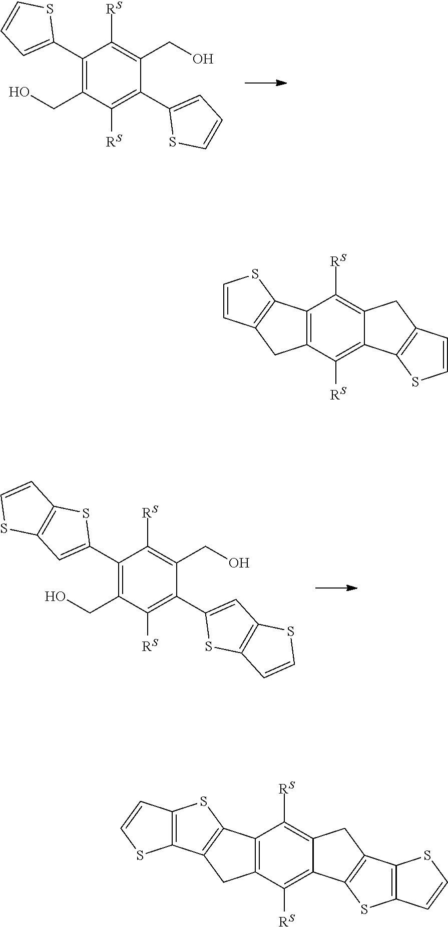

The advantages of the present process can be seen in its versatility, i.e. in the possibility to allow for a broad range of reactants and consequently a broad range of products that can be obtained by a very simple method. The present method also allows for rather easy upscaling from lab scale to commercial scales.

The products obtained from the present process are useful, for example, as components or precursors of materials for organic semiconductors, for organic photovoltaic cells, for organic light emitting diodes, to only name a few. Most importantly, these products are versatile building blocks or precursors of monomers for synthesizing advanced organic semiconducting materials.

### EXAMPLES

All reactants and solvents were obtained from commercial sources unless specified otherwise. 2,5-Bis-thieno[3,2-b]thiophen-2-yl-terephthalic acid diethyl ester was synthesized according to the synthesis published by C. Wang et al. in WO2013010614. 2,5-Dithien-2-yl-1,4-benzenedimethanol was prepared in the same manner as compound 1 by using 1,4-diethyl ester-2,5-di-2-thienyl-1,4-benzenedicarboxylic acid instead of 2,5-bis-thieno[3,2-b]thiophen-2-yl-terephthalic acid diethyl ester. 1,4-diethyl ester-2,5-di-2-thienyl-1,4-benzenedicarboxylic acid was synthesized according to the synthesis published by S. Chen et al. in Macromolecules, 2016, 49(2), 527-536. 2,5-Difluoro-3,6-dithien-2-yl-terephthalic acid diethyl ester was synthesized according to the synthesis published by M. D'Lavari et al. in WO2015154845. Biphenyl-2-yl-methanol was obtained from Sigma-Aldrich.

### Example 1

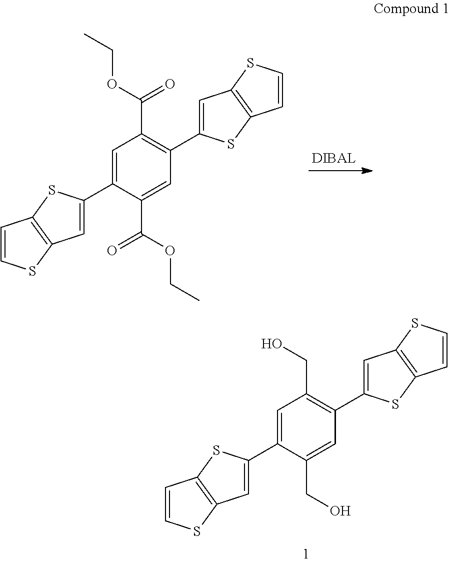 [SMILES: CCOC(=O)c1cc(-c2cc3sccc3s2)c(C(=O)OCC)cc1-c1cc2sccc2s1 ; OCc1cc(-c2cc3sccc3s2)c(CO)cc1-c1cc2sccc2s1]

To a mixture of 2,5-bis-thieno[3,2-b]thiophen-2-yl-terephthalic acid diethyl ester (25.2 g, 50.0 mmol) and anhydrous tetrahydrofuran (1000 cm³) at 0° C. was added diisobutylaluminum hydride (200 cm³, 250 mmol, 25% w/w in hexanes) dropwise over a period of 30 minutes. The reaction mixture was stirred at 0° C. for 4 hours and warmed slowly over 17 hours to 23° C. The reaction mixture was cooled to 0° C. and concentrated hydrochloric acid added until the mixture was acidic. The volatiles were removed in vacuo, the residue triturated with methanol (500 cm³) and the solid collected by filtration. The solid was washed with aqueous hydrochloric acid (100 cm³, 2%), methanol (100 cm³) and then recrystallised (tetrahydrofuran/methanol) to give compound 1 (19.4 g, 94%) as a yellow solid.

¹H-NMR (400 MHz, DMSO) 7.69-7.74 (6H, m), 7.49-7.51 (2H, m), 5.46 (2H, s), 4.69 (4H, s).

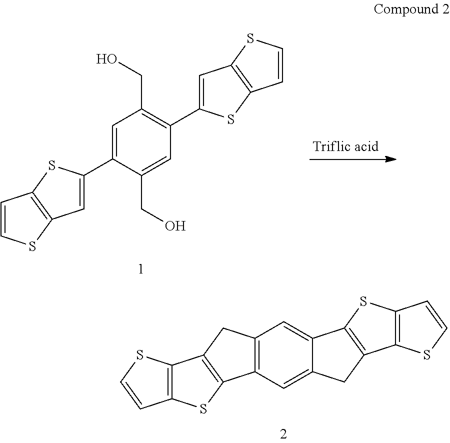 [SMILES: OCc1cc(-c2cc3sccc3s2)c(CO)cc1-c1cc2sccc2s1 ; c1cc2sc3c(c2s1)Cc1cc2c(cc1-3)Cc1c-2sc2ccsc12]

To triflic acid (10 cm³, 120 mmol) at −5° C. was added compound 1 (1.25 g, 3.02 mmol) in portions over 1 hour. The mixture was then stirred at −5° C. for 6 hours and warmed naturally with the cooling bath to 23° C. then stirred for 60 hours. The mixture was poured onto crushed ice (50 g) and the solid collected by filtration. The solid was washed with water (50 cm³), saturated aqueous sodium acetate (50 cm³), water (50 cm³) and methanol (50 cm³). The product was heated in boiling chlorobenzene (50 cm³) and the hot solution filtered. The solid was subjected to the extraction process a further three times and the filtrates combined. The solvent removed in vacuo to give compound 2 (0.71 g, 62%) as a yellow solid.

¹H-NMR (400 MHz, o-DCB, 120° C.) 7.38 (2H, s), 7.13 (4H, m), 3.60 (4H, s).

### Example 2

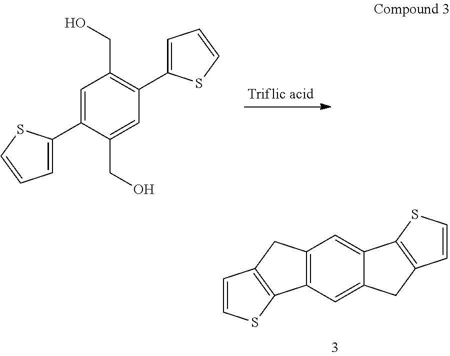 [SMILES: OCc1cc(-c2cccs2)c(CO)cc1-c1cccs1 ; c1cc2c(s1)-c1cc3c(cc1C2)-c1sccc1C3]

Triflic acid (30 cm³, 370 mmol) was cooled with an acetone-ice bath for 10 minutes (−7° C. external). 2,5-Dithien-2-yl-1,4-benzenedimethanol (1.51 g, 5.0 mmol) was added, in small fractions, to the stirred acid with cooling. The mixture was stirred with the cooling bath for 6 hours and then poured onto 100 g of crushed ice and the solid collected by filtration. The solid was washed with water (100 cm³), saturated aqueous sodium acetate (100 cm³), water (100 cm³) and methanol (100 cm³). The solid was boiled in chloroform (75 cm³) and then suction-filtered through a silica pad. The solvent was removed in vacuo to give compound 3 (277 mg, 21%) as a pale-yellow solid.

¹H-NMR (400 MHz, CDCl₃) 7.54 (2H, s), 7.22 (2H, d, J 4.9), 7.05 (2H, d, J 4.9), 3.67 (4H, s).

### Example 3

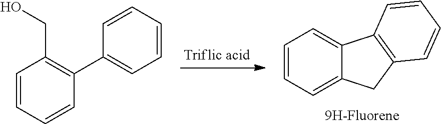

To triflic acid (30 cm³, 370 mmol) at −5° C. was added biphenyl-2-yl-methanol (1.5 g, 8.1 mmol) in portions over 1 hour. The mixture was then stirred at −5° C. for 6 hours and warmed slowly to 23° C. and then stirred over 17 hours. The mixture was poured onto crushed ice (50 g) and the solid collected by filtration. The solid was washed with water (50 cm³) and methanol (50 cm³) to give a pale yellow solid. GCMS of the crude yellow solid shows a peak at 4.37 mins (166 g/mol, 9H-fluorene) corresponding to a non-purified yield of 4%.

### Example 4

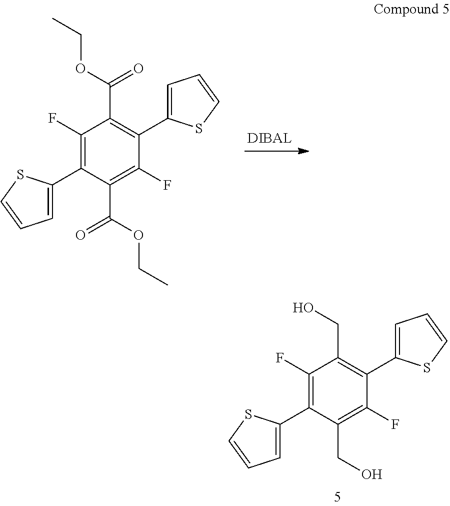 [SMILES: CCOC(=O)c1c(F)c(-c2cccs2)c(C(=O)OCC)c(F)c1-c1cccs1 ; OCc1c(F)c(-c2cccs2)c(CO)c(F)c1-c1cccs1]

To a solution of 2,5-difluoro-3,6-dithien-2-yl-terephthalic acid diethyl ester (2.00 g, 4.73 mmol) in anhydrous tetrahydrofuran (10 cm³) at −78° C. was added dropwise diisobutylaluminum hydride solution (23.7 ml, 23.7 mmol, 1 M in tetrahydrofuran) over 30 minutes. The reaction mixture was then allowed to warm to 23° C. and stirred for 17 hours. Hydrochloric acid (200 cm³, 2 M) was added slowly and the mixture stirred for 20 minutes. Concentrated hydrochloric acid (2 cm³) was added and the mixture stirred for a further 20 minutes. The product was extracted with diethyl ether (2×100 cm³) and the combined organics washed with water (100 cm³) and brine (100 cm³). The organic phase was then dried over anhydrous magnesium sulfate, filtered and the solvent removed in vacuo to give compound 5 (1.45 g, 91%) as an off-white solid. ¹H-NMR (400 MHz, DMSO) 7.81 (2H, dd, J 5.1 2.1), 7.38 (2H, dd, J3.5 1.2), 7.25 (2H, dd, J5.1 3.5), 5.32 (2H, t, J 5.0) 4.36-4.42 (4H, m).

¹⁹F-NMR (400 MHz, DMSO) −119.4.

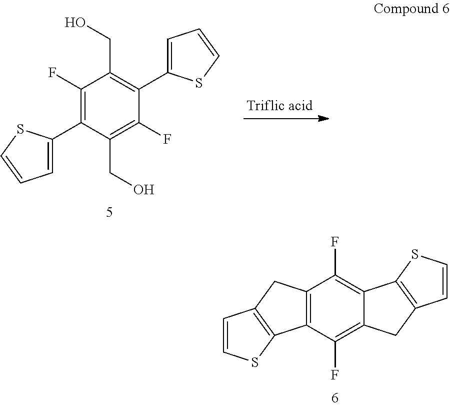 [SMILES: OCc1c(F)c(-c2cccs2)c(CO)c(F)c1-c1cccs1 ; Fc1c2c(c(F)c3c1-c1sccc1C3)-c1sccc1C2]

Triflic acid (14.5 cm³, 150 mmol) was cooled with an acetone-ice bath for 10 minutes (−7° C. external). Compound 5 (1.45 g, 4.3 mmol) was added, in small fractions, to the stirred acid with cooling. The mixture was warmed to 23° C. and stirred for 17 hours. The mixture was poured onto 100 g of crushed ice and the solid collected by filtration. The solid was washed with water (100 cm³), saturated aqueous sodium acetate (100 cm³), water (100 cm³) and methanol (100 cm³). The solid was boiled in chloroform (2×50 cm³) then collected by filtration to give compound 6 (1.07 g, 83%) as a brown solid.

¹H-NMR (400 MHz, CDCl₃) 7.43 (2H, d, J 4.9), 7.17 (2H, d, J 4.9), 3.86 (4H, s).

¹⁹F-NMR (400 MHz, DMSO-d₆) −131.7.

1. A process of reacting a reactant comprising two adjacent moieties Ar¹ and Ar², wherein
(i) Ar¹ and Ar² are linked by a carbon-carbon single bond;
(ii) Ar¹ and Ar² are at each occurrence independently selected from the group consisting of arenes, arenes substituted with R^{S}, heteroarenes and heteroarenes substituted with R^{S}, with R^{S} being a halogen or a carbyl group; and
(iii) one of Ar¹ and Ar² has a group of formula —CH₂—OH in ortho-position to said carbon-carbon single bond,
in the presence of a strong acid to obtain a product, wherein the Ar¹ and Ar² are fused to a five or six-membered ring, which comprises said carbon-carbon single bond and the formed CH₂-bridge.

2. The process according to claim 1, wherein the strong acid is triflic acid, polyphosphoric acid, fluorosulfuric acid, SbF₅, or BF₃, or a mixture comprising one or more of the above acids, or a mixture composed of a neutral solvent such as dichloromethane, chloroform and the above acids

3. The process according to claim 1, wherein the reactant comprises one or more structural unit of formula (I).

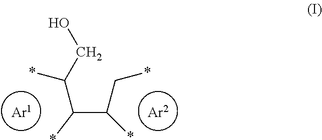

4. The process according to claim 1, wherein the reactant is selected from the group consisting of the following formulae (I-A), (I-B) and (I-C) 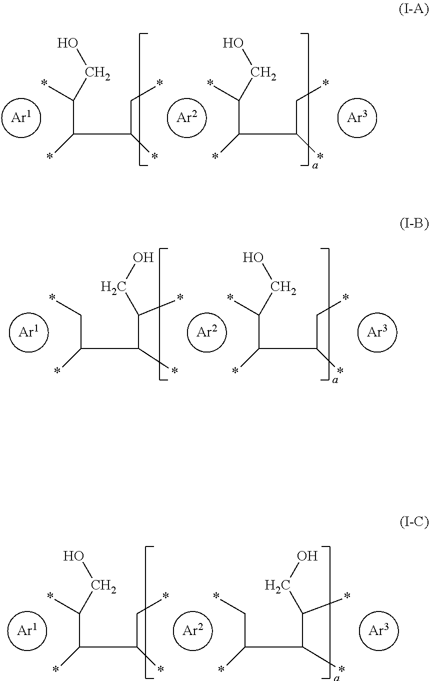
with Ar³ at each occurrence independently as defined as for Ar¹ and Ar²; a being an integer selected from the group consisting of 1, 2, 3, 4 and 5; and wherein adjacent —CH₂—OH groups may be cis or trans to one another.

5. The process according to claim 1, wherein the reactant comprises one or more structural units independently selected from the group consisting of the following formulae (I-A′), (I-B′), (I-C′), (I-D′), (I-E′) and (I-F′) 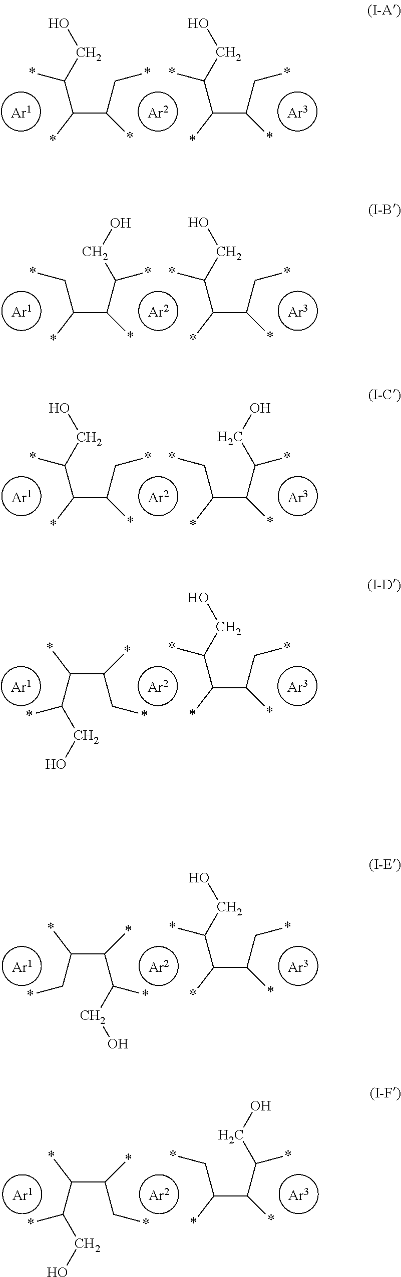
with Ar³ being as defined for Ar¹ and Ar².

6. The process according to claim 1, wherein Ar¹ and Ar² and—if present—Ar³ are at each occurrence independently selected from the group consisting of the following formulae (III-1) to (III-11) 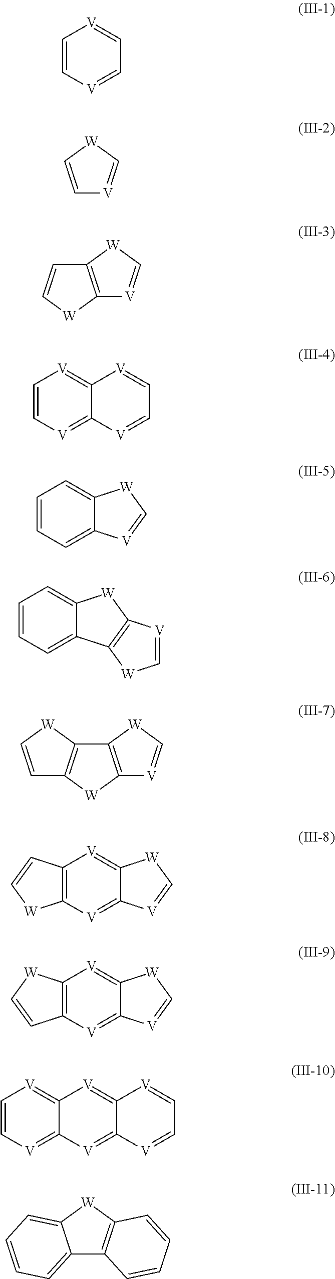
which may optionally be substituted with one or more group R^{S}, and wherein W is at each occurrence independently selected from the group consisting of S, O and Se; and V is at each occurrence independently CR⁰ or N, with R being at each occurrence independently selected from the group consisting of H, F, hydrocarbyl having from 1 to 40 carbon atoms and hydrocarbyl having from 1 to 40 carbon atoms wherein one or more hydrogen have been replaced by F.

7. The process according to claim 1, wherein R^{S} is at each occurrence independently selected from the group consisting of any group R^{T}, hydrocarbyl having from 1 to 40 carbon atoms wherein the hydrocarbyl may be further substituted with one or more groups R^{T}, and hydrocarbyl having from 1 to 40 carbon atoms comprising one or more heteroatoms selected from the group consisting of N, O, S, P, Si, Se, As, Te or Ge, with N, O and S being preferred heteroatoms, wherein the hydrocarbyl may be further substituted with one or more groups R^{T}, with R^{T} being at each occurrence independently selected from the group consisting of F, Br, Cl, —CN, —NC, —NCO, —NCS, —OCN, —SCN, —C(O)NR⁰R⁰⁰, —C(O)X⁰, —C(O)R⁰, —NH₂, —NR⁰R⁰⁰, —SH, —SR⁰, —SO₃H, —SO₂R⁰, —OH, —OR⁰, —NO₂, —SF₅ and —SiR⁰R⁰⁰R⁰⁰⁰, with R⁰, R⁰⁰ and R⁰⁰⁰ at each occurrence independently of each other selected from the group consisting of H, F, hydrocarbyl having from 1 to 40 carbon atoms, and hydrocarbyl having from 1 to 40 carbon atoms wherein one or more hydrogen have been replaced by F.

8. The process according to claim 1, wherein the reactant is 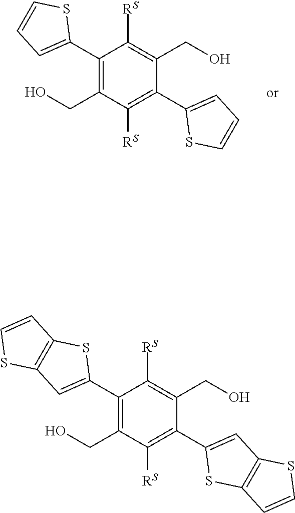
wherein R^{S} is H or F.

9. The process according to claim 1, wherein the molar ratio of triflic acid to the number of reactant —CH₂OH groups is at least 1.

10. The process according to claim 1, wherein the process is performed at a temperature of at most 50° C.
